(19) United States
(12) Patent Application Publication
(10) Pub. No.: US 2022/0279994 A1
(43) Pub. Date: Sep. 8, 2022

(54) Advanced Toilet Seat.

(71) Applicants: Jean-Baptiste P. Duprieu, New York, NY (US); James Keith Vanderpant, New York, NY (US)

(72) Inventors: Jean-Baptiste P. Duprieu, New York, NY (US); James Keith Vanderpant, New York, NY (US)

(21) Appl. No.: 17/740,018

(22) Filed: May 9, 2022

Related U.S. Application Data
(63) Continuation-in-part of application No. 17/193,894, filed on Mar. 5, 2021, now Pat. No. 11,326,332.

(51) Int. Cl. A47K 13/30; E03D 9/08; G01G 19/52

(52) U.S. Cl. CPC A47K 13/302; E03D 9/08; G01G 19/52; A61B 5/742

(57) ABSTRACT

This invention is an advanced toilet/bidet with a remote toilet seat that stores both the majority of the bidet hardware and an advanced software program, providing a toilet seat that is thinner and lighter than a traditional toilet/bidet combination. The toilet seat has a bidet nozzle, and an internal cavity in which additional hardware and software is stored. The toilet seat has sensors the record health-related data on each user and has a unique cleaning mechanism to ensures the highest sanitary standards.

### CROSS REFERENCE TO RELATED APPLICATIONS

This application is a Continuation-In-Part, and claims priority to, U.S. Utility application Ser. No. 17/193,894, entitled Advanced Toilet with Remote Bidet and Related Software Application, with a filing date of Mar. 5, 2021, the contents of which are incorporated by reference in its entirety.

### STATEMENT REGARDING FEDERALLY SPONSORED RESEARCH OR DEVELOPMENT

This invention was not federally sponsored.

### BACKGROUND OF THE INVENTION

### Field of the Invention

This invention relates to the general field of toilets, and more specifically, to a special toilet seat with unique biometric sensors and a related software application that can be attached to a standard toilet, providing a safer, cleaner, and healthier experience than that afforded by the prior art. The toilet seat sensors detect and record a number of biometric observations and provide feedback to a user.

### Brief Description of Invention

The invention comprises an advanced toilet seat which houses the majority of the traditional bidet hardware and also houses a software program. The invention can be hooked into an existing toilet with a water tank, where the hardware and software in the seat controls the bidet function, and additionally comprises electrical connections and an advanced software system that runs the device.

Enclosed within the seat is a water heater that takes water from a water source and heats it for the bidet portion of the invention. The heated water is then pushed through a vacuum breaker valve and solenoid valve before exiting through a flow meter. A mechanism that automatically opens and closes the toilet lid has a proximity sensor at the front of the toilet seat and a mechanical motor at the back of the toilet seat. Two speakers are used for audio features, a descale port allows for cleaning of heating components, and a drain.

On the bottom of the toilet seat are four sensors, each with a scale, with weight distribution being differently assessed on each sensor based on a profile of the user's normal stance on the toilet seat. In one embodiment, sensors with weight profiles of 38%, 32%, 16% and 12% is provided.

There currently exists toilet seats with bidets, but these toilet seats are generally large, heavy and bulky because of the extensive internal hardware needed to support both the toilet and bidet function. This creates a toilet seat that is expensive, and difficult to install/remove because of its size and weight. The current invention improves upon this problem by reducing the overall amount of components and also redesigning some of the larger internal components to custom fit into this design. This allows for a much thinner toilet seat design that is traditionally found in All-in-one toilets with built-in bidets, and can be used with existing toilets.

The prior art has several examples of bidets built into toilets, and even some toilets with various software applications, both there are either a) too bulky, b) too inefficient, or c) too expensive. Thus, there has existed a long-felt need for an efficient toilet that combines light weight, a thin profile, and is capable of obtaining and processing health-related data to help the users keep track of their health.

The current invention provides just such a solution by providing an advanced toilet seat/bidet that stores both the necessary hardware and an advanced software program, providing a toilet seat that is thinner and lighter than a traditional toilet/bidet combination. The toilet seat has sensors that record health-related data on each user and has a unique cleaning mechanism to ensure the highest sanitary standards.

### OBJECTS OF THE INVENTION

It is therefore an object of the present invention to provide a thin and lightweight toilet seat with a bidet function with numerous biosensing mechanisms.

A final object of the invention is to provide a software program that collects data from sensors on the toilet seat and provides the user many health-related measurements.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

One preferred form of the invention will now be described with reference to the accompanying drawings.

FIG. 1 is a side view of the invention.

FIG. 2 is a perspective view of the toilet seat showing the location of the sensors.

FIG. 3 is a cross-sectional view of the toilet seat.

FIG. 4 is a bottom, perspective view of the toilet seat.

FIG. 5 is a perspective view of the toilet seat showing the sensor placement.

FIG. 6 is a top, perspective view of the invention.

FIG. 7 is another perspective view of the toilet seat showing the location of the various components.

FIG. 8 is a bottom view of the toilet seat.

FIG. 9 is a perspective view of the deodorizer cartridge.

FIG. 10 is a bottom, perspective view of the toilet seat showing the sensor placement.

FIG. 11 is a bottom, perspective view of the drain portion of the invention.

FIG. 12 is another bottom, perspective view of the stool assessment portion of the invention

FIG. 13 is a flow chart showing the sequence of actions between the user and the invention.

FIG. 14 is a flow chart of one example of a user being assessed by the invention.

FIG. 15 is a flow chart detailing the sequence of a user using the invention.

### DETAILED DESCRIPTION OF THE FIGURES

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

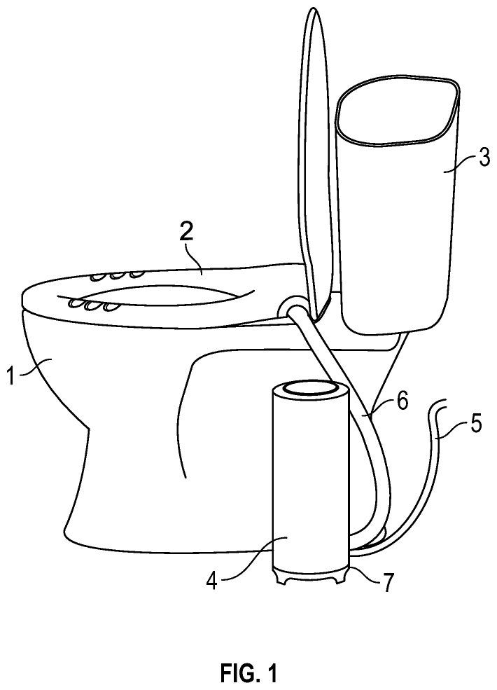

FIG. 1 is a side view of the invention. The toilet 1 has a toilet seat 2, into which sensors have been placed (not visible in this figure). A toilet tank 3 stores water and is connected to an external pod 4 by a wall to pod water hose 5. The pod 4, in turn, supplies water to the toilet bidet (not shown in this figure) through a pod to toilet water hose 6. The unit is powered by an electrical connection 7. By removing the hardware from a traditional toilet seat with a bidet, the invention provides a significantly lighter and thinner toilet, making it easier to install and remove.

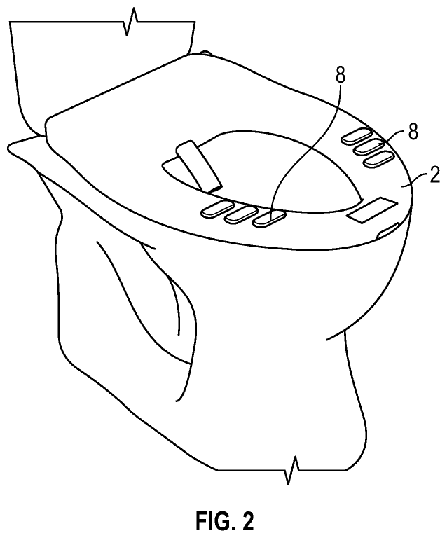

FIG. 2 is a perspective view of the toilet seat 2 showing the location of the sensors 8. The toilet seat has sensor pads 8 in at least two locations that take biometric data from a person sitting on the toilet. The design of this toilet allows for touch-free opening and closing, a heated seat, bidet, a control for the water temperature, deodorizer, sound masking, self-cleaning function, user recognition, and utilization of the advanced software system that runs the unit. The biometric sensors take measurements every time a person sits down, including but not limited to hydration levels, heart rate/HRV/ECG, body weight, BMI, body fat, muscle mass, and frequency of use tracking.

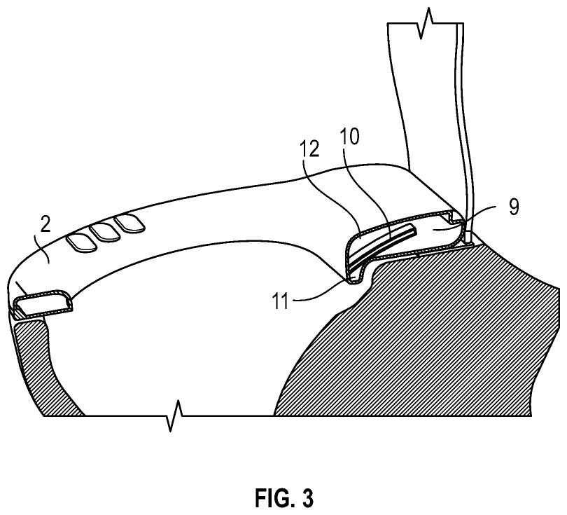

FIG. 3 is a cross-sectional view of the toilet seat. The bidet water nozzle 10 is hidden inside of toilet seat in a nozzle cavity in the under hang 11 of the toilet. There are additional cavities, 9 and 12, to house additional hardware to sit inside of the toilet rather than outside.

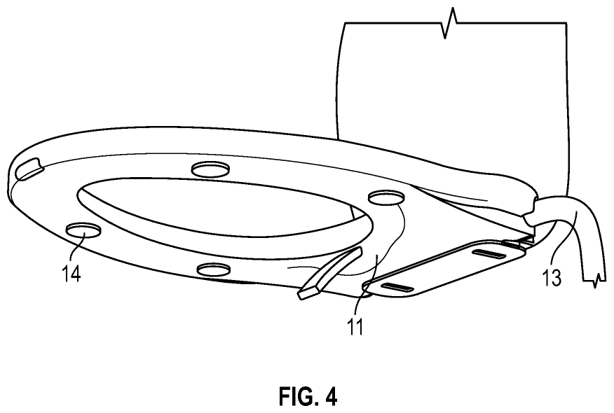

FIG. 4 is a bottom, perspective view of the toilet seat. This figure shows how the cleaning mechanism is stored in the actual seat ring, rather than in a base housing as is found in the prior art. The water supply 13 from the pod enters the assembly, and the inner cavities house all the hardware, leaving the under hang 11 clear from any electrical components. Silicon pads 14 help stability and also have scales/weight sensors to measure users' weight and weight distribution for user recognition.

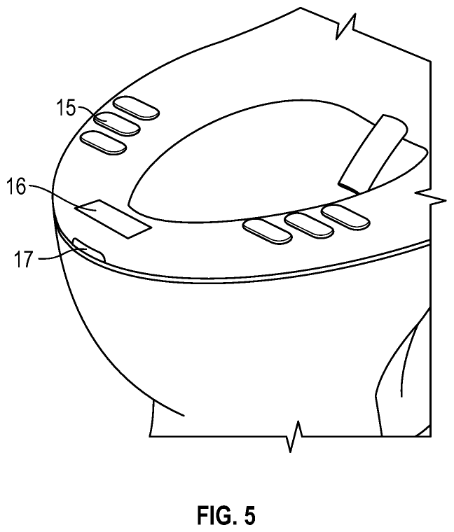

FIG. 5 is a perspective view of the toilet seat portion of the invention. The bottom of the seat can feature either matrix displays 16 or flexible, bendable OLED/LCD under the top surface of the seat with can be activated by the microcontroller. The seat also has Front Sensors 17 in the front of the seat to detect the user's presence. When the user is detected as approaching the seat the lid will automatically open. Also used to detect when no user is present for a certain amount of time and will automatically close.

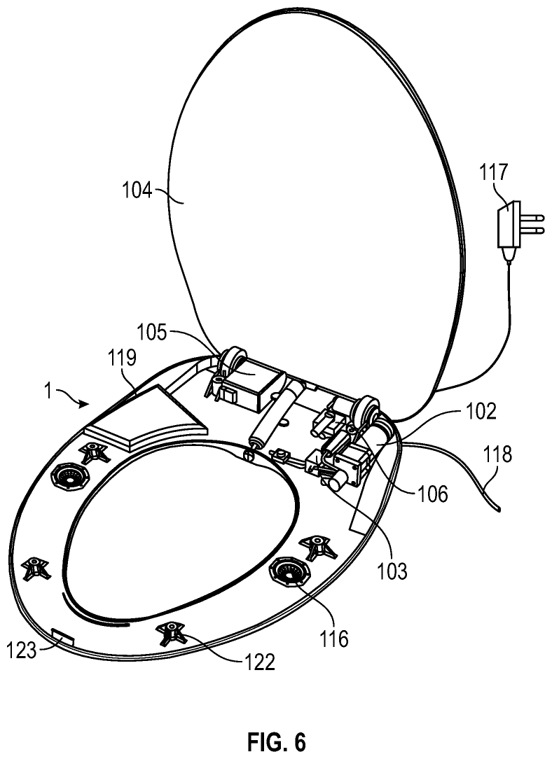

FIG. 6 is a top, perspective view of the invention. Electricity to the unit is supplied through an electrical connection 117. Water is supplied to the unit via a water source 118. Once water enters the toilet seat, generally 101, it is heated in a water heater 102, then directed to a nozzle 121 via a vacuum breaker valve 106 and a solenoid valve 103, with the volume of water controlled via a flow meter 107. Next to the nozzle 121 is a stool sensor 120 which takes biofeedback readings of a stool being dropped into the toilet. The toilet lid 104 is raised and lowered automatically by an auto open/close mechanism which has a motion detector 123 on the front and a motor 105 on the back such that the user need only remotely signal the device to open the toilet lid, thereby adding to the sanitation of the use. In this embodiment of the invention, two speakers 116 give audio feedback to the user which several silicon pads 122 keep the cavity between the toilet seat top and the toilet sea bottom well-supported. Capacitive touch sensors (8 in FIG. 2) reach up through the top of the toilet seat and collect data from the user at various points. Software is housed in a software housing 119.

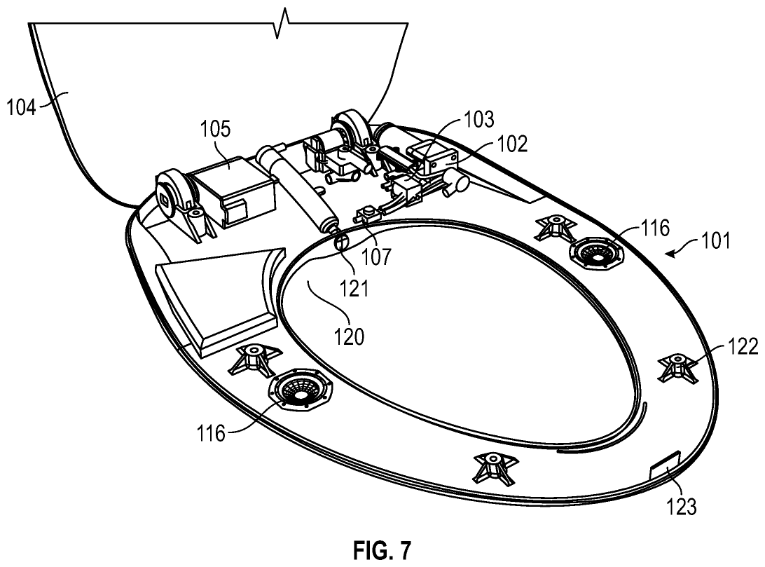

FIG. 7 is another perspective view of the toilet seat showing the location of the various components. The toilet lid 104 is automatically raised and lowered without having the user touch the toilet seat via a remote control which can, in one embodiment, activate the motor 105 to raise the toilet lid. After use, the invention senses that the user has finish using the toilet and the motor retracts the lid 104 over the toilet, and the motion detector 123 activate the motor 105 which then disengagably latches the lid 104 to the toilet seat, generally 101.

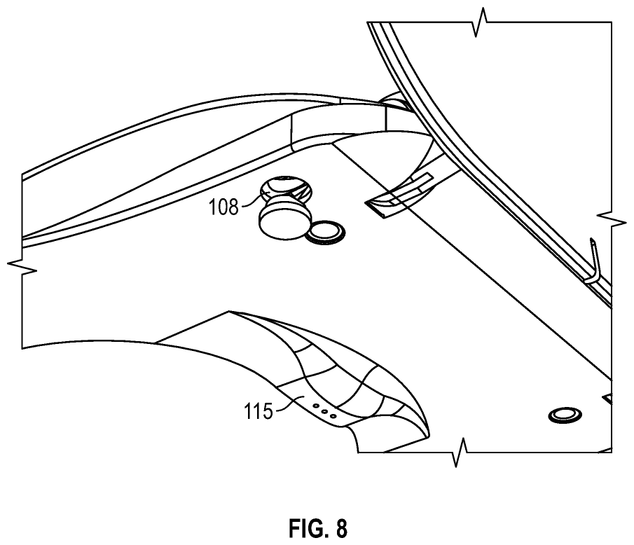

FIG. 8 is a bottom view of the toilet seat. A descale port 108 allows for the system to be flushed as needed to keep contaminants and build-up out of the invention and a drain 115, allows for any water that enters the inside cavity to drain out.

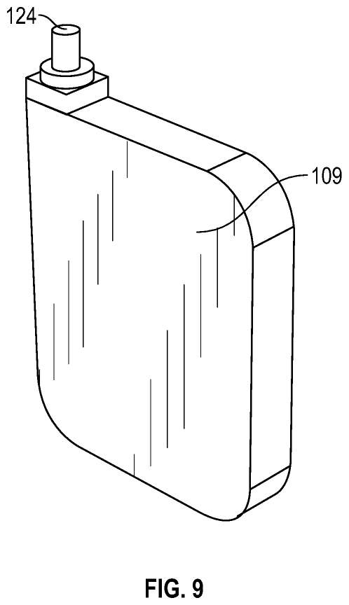

FIG. 9 is a perspective view of the deodorizer cartridge 109. The deodorizer cartridge is attached to the water flow after it leaves the water source and before it exits the nozzle, and deposits a small amount of deodorizer into each batch of water before it enters the toilet. The deodorizer cartridge has an engagable tip 124 that can be removed from the invention when the deodorizer cartridge is empty, and a new cartridge can be inserted.

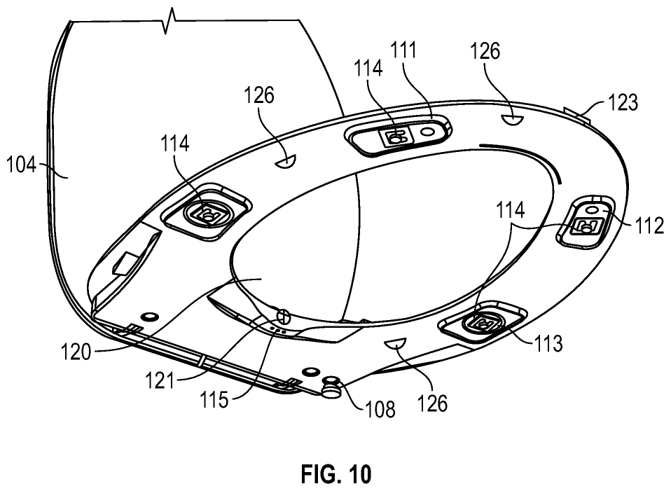

FIG. 10 is a bottom, perspective view of the toilet seat showing the sensor placement. In the embodiment shown in this drawing, there are four sensors placed at various locations around the bottom of the toilet seat. Each has a scale 114. In this figure, a first sensor 110 detects 16% of the person's weight, a second sensor 111 detects 38%, a third sensor 112 detects 32% and a fourth sensor detects 14%. The invention can then calculate a number of factors about the user, and put this data into its memory, so that it will recognize that same person the next time he or she uses the toilet by the weight distribution and other factors. The descale port 108, drain 115, stool sensor 120 and nozzle 121 are also visible in this figure. LED's 126 on the bottom of the toilet seat are triggered by the lid opening to turn on and illuminate the toilet bowl.

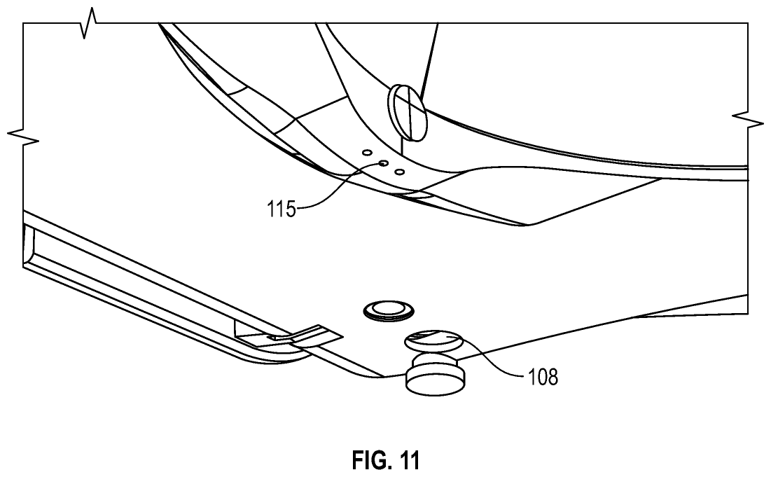

FIG. 11 is a bottom, perspective view of the drain portion 115 of the invention as well as the descale port 108.

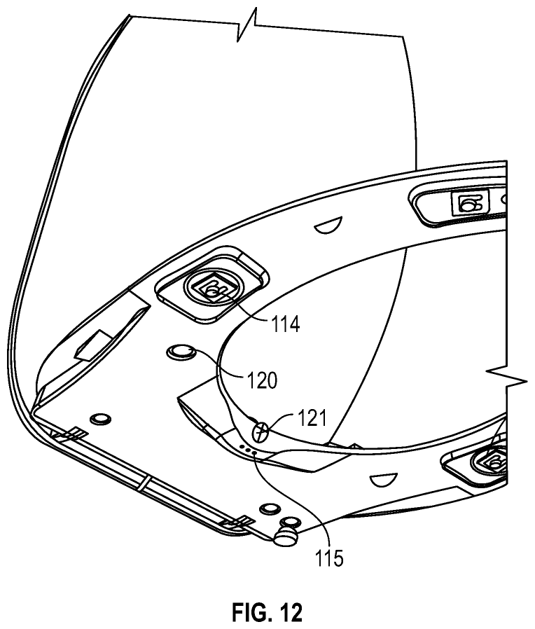

FIG. 12 is another bottom, perspective view of the stool assessment portion of the invention.

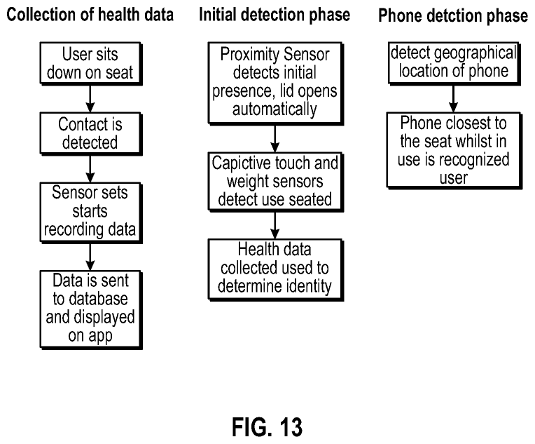

FIG. 13 is a flow chart showing the sequence of actions between the user and the invention. The seat sensors and software coordinate to begin assessing the user as soon as the user sits down on the seat. Once contact is detected, the sensors start recording date. The data is then sent to the database, analyzed by the software, and displayed on an app. The app can be downloaded by the user on a cell phone and/or displayed on a remote screen in the bathroom.

Once the user has used the toilet before, his/her data is stored and the next time he/she uses the toilet a proximity sensor detects the presence of a person and automatically opens the lid (through motor 105 as illustrated in other figures). Once the lid open, the user sits down and capacitive touch sensors (125 in other figures) and weight sensors (114 in other figures) detect the identity of the user. In addition to detecting the user, the software records any changes in the biofeedback received through the toilet seat on the user and notifies the user of any important changes that it detects.

There is also a phone detection element of the invention which detects the geographical presence of nearby cell phones, and hones in on the nearest cell phone in the bathroom, and uses that to confirm the identify of the user.

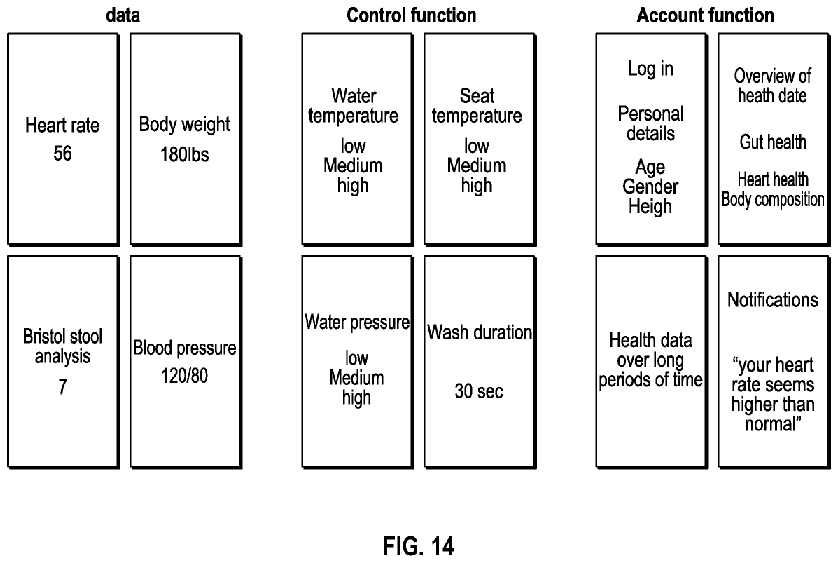

FIG. 14 is a flow chart of one example of a user being assessed by the invention, and how it appears on the app. In this particular example, the data collected includes the heart rate, body weight, blood pressure, and a Bristol stool analysis. A control function is also assessed, including the water temperature, seat temperature, water pressure, and the wash duration. All this data is fed to the software, which, after a successful log in, to ensure security, provides the user with a large amount of useful data related to his/her particular health at the time of use as well as identifying any promising or dangerous trends in the user's health.

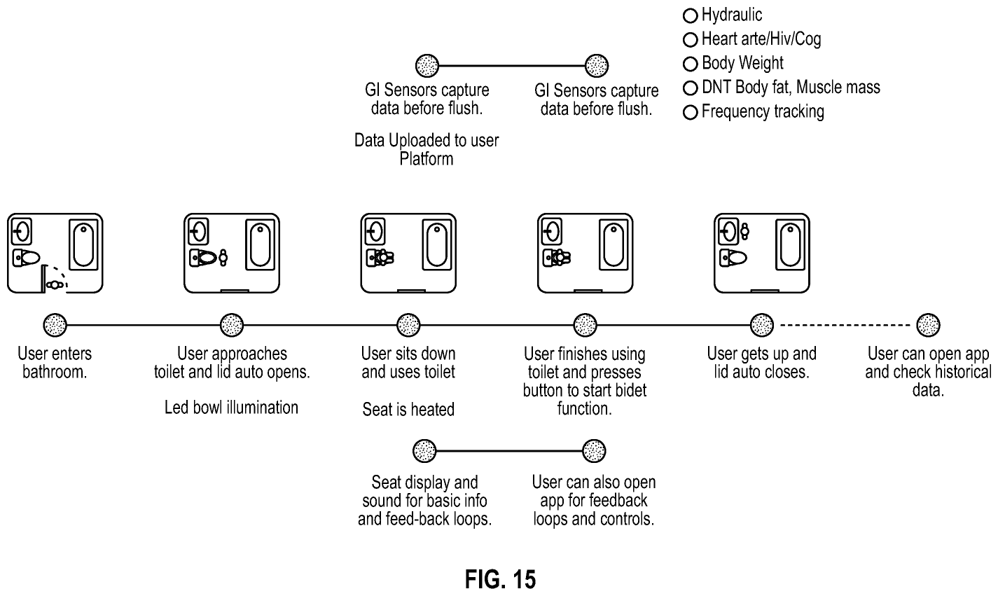

FIG. 15 is a flow chart detailing the sequence of a user using the invention. The user enters the bathroom and the phone detector tries to recognize the user. As the user approaches the toilet, the user's presence and apparent desire to the use the toilet is detected, and the lid automatically opens, and the blow is illuminated with an LED. The user then sits down on the toilet and the seat is immediately warmed up. The sensors begin gathering data and using both the stance of the user as well as biofeedback from the sensors to confirm the identity of the user. After the user finishes, he/she presses the sensors to confirm the identity of the user. After the user finishes, he/she presses the bidet function and then gets up to leave, at which point the device detects the user leaving and automatically closes the lid.

All of the material in this patent document is subject of copyright protection under copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. A toilet seat assembly, comprising a toilet seat, where the toilet seat has a top surface and a bottom surface, with a cavity exists between the top surface and the bottom surface, an electrical connection, a water-source-to-toilet seat hose, where the electrical connection provides power to the toilet seat, where the water-source-to-toilet seat hose connects a water source to the toilet seat, where the toilet seat comprises at least one sensor, where the toilet seat houses both a hardware component and a software component, where the hardware component comprises a water heater, an Electrical Engineering—Mechanical control outline, a flow meter, a solenoid valve, a speaker, a drain, a descale port, a stool sensor, at least one LED light, and a deodorizer bottle, where the at least one sensor sends at least one data to the software component, where the software component performs at least one calculation and displays the at least one calculation on a viewing device, where the cavity contains one or more internal parts are stored, where the cavity is not visible from outside of the toilet assembly, where the cavity houses a nozzle, where the nozzle is fed water from the water-source-to-toilet hose, where the nozzle protrudes from an underside of the toilet seat, and delivers a stream of water, where the cavity additionally comprises a vacuum breaker valve, where the deodorizer container is connected to the water-source-to-toilet hose, such that a quantify of deodorizer is added to a quantity of water moving from the water source to the nozzle, where the toilet seat comprises one or more silicon pads, where each of the one or more silicon pads is located in a an underside location on the toilet seat, where each of the one or more silicon pads has a scale and a weight distribution sensor such that each of the one or more silicon pads can measure a user's weight and determine how that user's weight is distributed in a weight distribution assessment, where the software component uses the user's weight and the weight distribution assessment to identify the user, where the toilet seat additionally comprise one or more sensor pads, where each of the one or more sensor pads is located in a top, front position on the toilet seat, where each of the one or more sensor pads records at least one item of biometric information, where the toilet seat comprises at least two sensors, where the at least two sensors are biosensors, where the toilet seat additionally comprises at least one dot matrix display, where each dot matrix display is located inside the toilet seat, where the toilet seat additionally comprises one or more front sensors, where each of the one or more front sensors is located on a front section of the toilet seat, where each of the one or more front sensors detects when a potential user approaches the toilet seat, and causes a toilet lid to automatically open, and, can also detect when there are no potential users in the vicinity and cause the toilet lid to automatically close after passage of a certain time period through an automatic open/close mechanism, where the automatic open/close mechanism has a retractable clip on a toilet seat front, and a mechanical lifting/closing mechanism located in a cavity back near the toilet lid.

2. The toilet seat assembly of claim 1, where the software component comprises an initial detection phase, a phone detection phase, a sex-of-user detection phase, a collection of health data phase, a computation of health data phase, and a display of data, and additionally comprising a hand-held digital display device, where the hand-held digital display device has, at a minimum, a health function, a control function, a store function, and an account function.

3. A toilet seat assembly, comprising a toilet seat, a toilet seat, an electrical connection, a water-source-to-toilet seat hose, where the electrical connection provides power to the toilet seat, where the water-source-to-toilet seat hose connects a water source to the toilet seat, where the toilet seat is an external toilet seat.

4. The toilet seat assembly of claim 3, where the toilet seat comprises at least one sensor.

5. The toilet seat assembly of claim 4, where the toilet seat additionally comprises an internal cavity, a water nozzle, an under-hang component, and a vacuum breaker.

6. The toilet seat assembly of claim 5, where the toilet seat houses both a hardware component and a software component, where the hardware component comprises a water heater, an Electrical Engineering—Mechanical control outline, a flow meter, a solenoid/regulator, a speaker, a ground connection for water, a drain, a descale port, a stool sensor, and a deodorizer bottle.

7. The toilet seat assembly of claim 6, where the toilet seat is an external toilet seat and is attachable to an existing toilet.

8. The toilet seat assembly of claim 7, where the at least one sensor sends at least one data to the software component, where the software component performs at least one calculation and displays the at least one calculation on a viewing device.

9. The toilet seat assembly of claim 4, where the toilet seat comprises a cavity, where one or more internal parts are stored, where the cavity is not visible from outside of the toilet assembly.

10. The toilet seat assembly of claim 9, where the cavity houses a nozzle, where the nozzle is fed water from the water-source-to-toilet seat hose, where the nozzle protrudes from an underside of the toilet seat and delivers a stream of water.

11. The toilet seat assembly of claim 10, where the cavity additionally comprises a vacuum breaker valve.

12. The toilet seat assembly of claim 11, where the toilet seat additionally comprises a deodorizer container, where the deodorizer container is connected to the toilet seat-to-toilet hose, such that a quantify of deodorizer is added to the quantity of water moving from a water pump to the nozzle in the water-source-to-toilet seat hose.

13. The toilet seat assembly of claim 4, where the toilet seat comprises a stool sensor and one or more silicon pads, where each of the one or more silicon pads is located in a front, underside location on the toilet seat, where each of the one or more silicon pads have a scale and a weight distribution sensor such that each of the one or more silicon pads can measure a user's weight and determine how that user's weight is distributed in a weight distribution assessment.

14. The toilet seat assembly of claim 13, where the software component uses the user's weight and the weight distribution assessment to identify the user.

15. The toilet seat assembly of claim 14, where the toilet seat additionally comprises one or more sensor pads, where each of the one or more sensor pads is located in a top, front position on the toilet seat, where each of the one or more sensor pads records at least one item of biometric information.

16. The toilet seat assembly of claim 15, where the toilet seat additionally comprises at least two sensors, where each of the at least two sensors are biosensors.

17. The toilet seat assembly of claim 4, where the toilet seat additionally comprises at least one dot matrix display.

18. The toilet seat assembly of claim 17, where each dot matrix display is located inside the toilet seat.

19. The toilet seat assembly of claim 4, where the toilet seat additionally comprises one or more motion detector sensors, where each of the one or more motion detector sensors is located on a front section of the toilet seat, where each of the one or more motion detector sensors detects when a potential user approaches the toilet seat, and causes a toilet lid to automatically open, and, can also detect when there are no potential users in the vicinity and cause the toilet lid to automatically close after passage of a certain time period.

20. The toilet seat assembly of claim 19, where the software component comprises an initial detection phase, a phone detection phase, a sex-of-user detection phase, a collection of health data phase, a computation of health data phase, and a display of data, and additionally comprising a hand-held digital display device, where the hand-held digital display device has, at a minimum, a health function, a control function, a store function, and an account function.
